(19) United States
(12) Patent Application Publication
(10) Pub. No.: US 2003/0196661 A1
(43) Pub. Date: Oct. 23, 2003

(54) Power inhaler employing unit doses of powders in pressurized packages

(76) Inventors: Richard George Miekka, (US); Richard A. Fotland, (US)

Correspondence Address: RICHARD A. FOTLAND, 1 CRAB APPLE LANE, FRANKLIN, MA, 02038, US

(21) Appl. No.: 10/125,090

(22) Filed: Apr. 19, 2002

(51) Int. Cl. A61M 15/00

(52) U.S. Cl. 128/203.21

(57) ABSTRACT

A process for producing uniform small doses of finely divided substances that consists of metering precise quantities a material onto the surface of a containment vessel, pressurizing the containment vessel with a liquefied gas, and sealing the containment vessel while pressurized. A major application involves the packaging of fine pharmaceutical powders into small unit doses for inhalation therapy. Liquid nitrogen is a preferred liquefied pressurizing agent. A liquefied noble gas may be employed as a dispersing medium for sensitive or highly reactive substances, and simple hydrocarbon gasses may be used where flammability and reactivity are not problems. If the liquefied gas is used as a dispersing medium, the containment vessel may be sealed prior to the total evaporation of the liquid. Alternatively, the liquefied gas may be metered into the containment vessel after the substance has been deposited onto the surface of the containment vessel, but prior to its sealing. The containment vessel is thus pressurized to relatively high pressures to facilitate the subsequent aerosolization of the pharmaceutical in an inhaler during inhalation therapy.

This invention provides a method for aerosolizing uniform small doses of finely divided substances and more particularly, the invention relates to a technique employing liquefied gases for pressurizing packages of fine pharmaceutical powders which, when ruptured, rapidly disperse the powder into an air stream for inhalation.

### BACKGROUND OF THE INVENTION

The therapy of lung disease often relies on inhaled medications. Bronchodilators have are widely employed in the treatment of asthma. Inhaled aerosolized antiviral agents are employed in the treatment of infectious diseases. Although most inhaled medications are given for their local effect, there is much recent interest in aerosol delivery of medications for systemic absorption. Inhaled drugs, in the form of very small dry powder particles, may be rapidly and directly absorbed into the blood stream. Thus, for example, proteins and peptides may be self-administered rather than administered by injection.

The list of drugs currently under investigation for inhalation delivery is quite extensive. Aerosolized insulin for diabetes is anticipated to become a major application of inhalation therapy.

Most large organic molecules, including proteins and peptides, are denatured by stomach acid when ingested. Absorption in the peripheral parts of the respiratory system overcomes this problem. Thus, the physician has means to provide the patient with a technique whereby the patient may self-administer large molecule medicaments without injection. The value of inhalation therapy in administering insulin, for example, is obvious.

Prior to the development of dry powder inhalers, most inhalation therapies employed canisters of pressurized chlorofluorocarbon propellants to disperse drugs. Environmental concern relating to CFC destruction of the earth's ozone layer has reduced the utility of this approach.

Dry powder inhalers for pulmonary drug delivery require dose levels that range from 15 micrograms to over 1,000 micrograms. Powder particle mean diameters of between 0.5 and 5.0 microns are required to provide effective deposition within the lung since larger particles tend to deposit in upper airways without any useful absorption to the circulatory system.

It is difficult to provide metered doses within the required tolerances at the 15 to 250 microgram levels. High-speed weighing systems are generally limited to dose sizes of about 5,000 micrograms or greater and thus require the active pharmaceutical be diluted with an excipient, such as lactose powder, in order to increase the total measured mass. This dilution approach is subject to limitations in mixing uniformity and the aspiration of extraneous matter by the patient.

Another approach for low dose packaging involves dispersing the active powder in a medium that is in a liquid state at room temperature and pressure. The packaging substrate is then filled or coated and the liquid evaporated leaving the powder residue on the surface of the substrate. This approach has limitations in view of potential chemical reactions between the pharmaceutical medicament and the dispersing solvent. Government agency approvals are often required for the use of this process because of these potential interaction problems.

Yet another approach for low dose packaging involves the electrostatic precipitation of aerosolized medicament onto the surface of the medicament package. Abrams et al, U.S. Pat. No. 5,699,649, describe a system employing an endless belt that is charged, developed with an aerosolized powder, and the powder image then transferred to the package. The direct electrostatic precipitation of aerosolized powder is disclosed in Pletcher, U.S. Pat. No. 5,669,973. An improvement in this electrostatic precipitation apparatus is described by Pletcher et al, U.S. Pat. No. 5,714,007. These electrostatic deposition techniques require complex control equipment to accurately meter the required dosage into each package. The rate of powder deposition is also limited due to particle transit time effects and limitations in the mass density of the aerosol. Difficulties in re-aerosolizing the particles in the user's inhaler, because of the large electrostatic forces on the charged particles, may also be significant.

Many problems relate to the delivery of dry powder pharmaceuticals to the patient. Several devices rely on inhalation by the patient to provide the power to aerosolize the powder. Young patients, older patients, or patients with asthma often do not have the capacity to strongly inhale. Other inhalers rely on external sources of pressurized gas to disperse or aerosolize a powder. Vaghefi, U.S. Pat. No. 5,875,776, employs a gas cylinder to supply the energy required to rupture a sealed dosing cartridge. He also describes an alternate approach employing spring-loaded plungers to penetrate the airtight cover of dosing cartridges.

The present invention provides a cost-effective method for repeatedly filling unit dose packages with accurate masses of fine powder medicament under high pressures of non-reactive gas for subsequent rapid aerosolization in inhalers thus eliminating the requirement for an external aerosolizing power source.

### SUMMARY OF THE INVENTION

The invention provides a process for inhalation therapy employs a pressurized unit dose package. The packaging process involves uniformly mixing a pre-weighed amount of finely divided substance with a known volume or known weight of liquid nitrogen. A uniform dispersion of the powder in the liquid nitrogen medium is obtained after mixing. Small volumetric measures of liquid may then be drawn from the liquid reservoir and deposited in the unit dose package. The package is sealed before all of the nitrogen has evaporated so that, after completion of evaporation, the container is pressurized. Alternatively, the liquefied nitrogen may be allowed to evaporate to dryness and a metered portion of a liquefied gas metered into the package, which would then be sealed prior to the liquid evaporation. The metered liquid dispersions may be deposited into any of a wide variety of individual capsules, blister packs, or other forms of packaging pre-forms. Metering and controlling the filling volume may be carried out using any of a number of well known high-speed filling line systems. A preferred method employs liquid nitrogen although, alternately, other liquefied gases may be employed.

Our co-pending application Ser. No. 09/427996, teaches the use of liquid nitrogen dispersions to provide very uniform small doses of powder packages for inhalation therapy. This method of this invention extends the utility of employing liquid nitrogen to provide a pressurized package for rapid aerosolization of medicament powders in inhalation therapy.

Inhalation therapy is carried out by loading a unit dose into an inhaler and then puncturing, slitting, or otherwise rapidly opening the pressurized package. The high velocity turbulent gas aerosolizes the medicament powder within a chamber in the inhaler and the resultant aerosol inhaled to deliver the powder to the lung.

The advantages of employing liquid nitrogen in this application include:

1. Liquid nitrogen is chemically inert both because of its chemical makeup and extremely low temperature.

2. Liquid nitrogen is very low in cost; high volume prices are about twenty cents per pound.

3. Liquid nitrogen has a low viscosity, which is of value in processing and mixing.

4. Liquid nitrogen has a very low dielectric constant, which is useful in stabilizing dispersions.

5. The extremely low temperature of liquid nitrogen allows powders to be comminuted without fear of generating high temperatures, which might change the properties of the active ingredient.

6. At room temperature, liquid nitrogen rapidly evaporates.

7. The nitrogen gas realized from the evaporation of the liquid phase may be employed as an inert package atmosphere in the final product.

8. Because of the inert nature of the liquid, the active pharmaceutical may be stored for long periods of time prior to packaging.

9. Technologies for liquid nitrogen shipping, handling, storing, and carrying out processing operations, such as cryogenic grinding, are well developed.

10. A unit dose package may be sealed while still retaining a small amount of liquid nitrogen. As the package is warmed, the liquid nitrogen evaporates to pressurize the sealed package with an inert gas. The high pressure is valuable for re-aerosolizing the powder in the package when used in an appropriate inhaler.

In one embodiment of the invention, a known small mass of fine powder medicament is introduced into a known mass of liquid nitrogen that is contained in a Dewar cryostat. The mixture is stirred until the medicament powder is well dispersed. A small metered quantity of the dispersion is then introduced into a packaging blister. This operation is repeated to sequentially fill an assembly of blister packs. The liquid nitrogen is allowed to partially evaporate and the blisters sealed prior to the complete vaporization of the liquid nitrogen. A high pressure then develops in the package as the liquid nitrogen remaining in the package vaporizes. The user would insert the pressurized pack into his inhaler. During inhalation, the user would actuate a mechanism that ruptures the pack thus ejecting the powder, now completely aerosolized, into the air stream being inhaled.

### BRIEF DESCRIPTION OF THE DRAWINGS

FIG. 1 shows a flow diagram illustrating material flow.

FIG. 2 depicts a typical liquefied gas fill line.

FIG. 3 shows an inhaler employing a pressurized unit dose package.

FIG. 4 shows a sealed tube containing powder.

FIG. 5 shows a packaging method employing a capsule.

### DETAILED DESCRIPTION

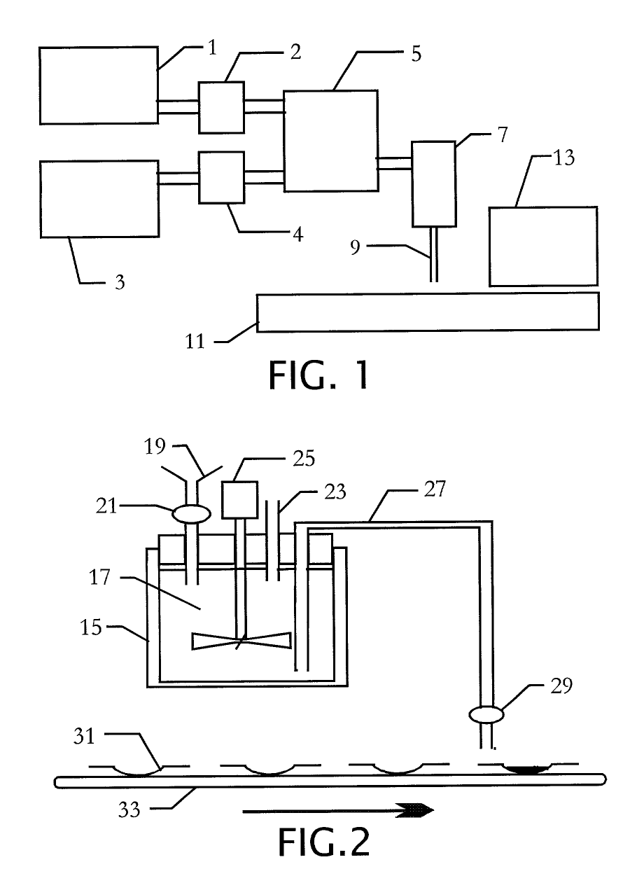

The general concept of the finely divided substance packaging process according to the present invention is schematically diagramed in FIG. 1. The finely divided substance filling system consists of a storage cryostat 5 in which the finely divided substance is mixed with a liquefied gas, typically liquid nitrogen, to form a uniform dispersion and stored pending the package fill operation. The liquefied gas is supplied through metering valve 2 from reservoir 1. A finely divided substance source 3 is employed as a reservoir of the finely divided substance that measured out employing metering device 4 for delivery to mixing cryostat 5. Filling line 11 sequentially transports substrates that are to receive a metered dose of dispersed powder under fill nozzle 9. Each metered dose is dispensed under control of metering valve 7. Post filling operations such as liquid medium evaporation, sealing, labeling, etc are carried out at filling line station 13

This fill system may operate in a batch mode. In this case, a measured quantity of liquefied gas together with a measured quantity of finely divided substance is periodically introduced into cryostat 5. Alternately, continuous feeds may be employed such that a constant volume of the fill mixture is maintained in cryostat 5.

The liquefied gas reservoir 1 is typically pressurized thus forcing the liquid through metering device 2. The handling and storage of liquefied gases as well as instrumentation for monitoring and controlling flow, temperature, liquid level, etc are well known. The Handbook of Cryogenic Engineering, edited by J. G. Weisend II, published by Taylor & Francis, 1998 describes many of these techniques in detail.

Finely divided substance stored in reservoir 3 may be fed using a metering device 4 in the form of a screw feeder either in a controlled continuous delivery or in an intermittent fashion. Alternately, metered feed device 4 may consist of a platform weighing mechanism that is loaded with a predetermined mass of powder and then fed, using vibration of the weighing platform to feed a measured mass of powder into a screw feeder that communicates with cryostat 5. More than one finely divided substance may be introduced into cryostat 5 when desired. Two or more materials may be premixed and added to reservoir 3 or, alternately, several reservoirs and metering devices may be employed in parallel to feed powders. In this case, it is very simple to modify the amounts of each added powder on a batch-by-batch basis.

Size reduction of the dispersed powder may be carried out without thermal damage to the powder in view of the low process temperature provided by employing a liquefied gas as the dispersing medium. This size reduction may be carried out in cryostat 5. The high pressure generated by evaporating cryogenic fluid may be employed to force the dispersion through a high-pressure valve homogenizer. Individual biological cells may be ruptured in this manner thus releasing proteins and enzymes from such cells. Alternately, size reduction may be carried out by employing a cryostat in the form of a stirred bead or a ball mill. Vibratory bead or ball mills may also be employed in this regard. These size reduction equipments are described in Perry's Chemical Engineers' Handbook, Seventh Edition, McGraw-Hill, Ch. 20 (1997). In addition to size reduction, the cryostat may also include filtering apparatus so that only selected particle size materials are introduced into metering valve 7.

Transport 11 operates intermittently to sequentially position receptor substrates 31 under fill nozzle 9. The unit dose receptor substrates may be in the form of an unsealed capsule, blister pack, film or foil, tube, or vial. Receptor substrates 31 are cooled prior to the filling operation in order to eliminate spattering of the filling mixture due to rapid evaporation of the liquefied medium. After filling, the filled receptor moves to sealing station 13 where the filled package is sealed. The sealing process depends upon the package configuration and material of construction.

A sufficient time interval is provided between time of filling and time of sealing to evaporate a small portion of the liquefied gas. If all of the liquid is evaporated, a metered amount of the same liquefied gas, or a different liquefied gas is introduced just prior to sealing. In some instances, it may be necessary to employ a noble gas such as argon or krypton as the pressurizing gas in order to prevent the degradation of extremely unstable substances. The desired package pressure range depends upon the inhaler design, package opening parameters such as opening rate and opening size, and the aerosolizability of the medicament powder. In general, gauge pressures of between about 20 pounds per square inch and 300 pounds per square inch are employed.

The packaging method of the present invention may be employed with a variety of pharmaceuticals. Some contemplated examples illustrating the application range include the following: analgesics, sedatives, antianginal agents, antianxiety agents, antipsychotic agents, antiarrhythmics, antiarthritic agents, anticoagulants, anticarcinogens, thrombolytic agents antifibrinolytic agents, anticonvulsants, antiparkinson agents, antideprssants, antihistamines, antihypertensive agents, antibacterial agents, antifungal agents, antiviral agents, diabetes treatment agents, cancer chemotherapy agents, antimicrobials, anti-infectives, bronchodialators, hormones, hypoglycemic agents hypolipidemic agents, proteins, peptides, nucleic acids, specialized cells, antiulcer agents, antireflux agents, antinauseants, and the like. While the major application area involves pharmaceuticals, other potential applications of the present invention include packaging of specialty chemicals. Semiconductor doping agents, for example, may be packaged in precise small quantities. Powerful and expensive chemicals such as root growth hormone, chemical catalysts, and the like may be packaged using the method of the present invention.

Liquids and active substances dissolved or dispersed in a liquid may also be packaged or dispensed employing the method of the present invention. In this case, fill nozzle 9 is terminated with a spray nozzle. The substance to be packaged or dispensed is now sprayed directly into the liquefied dispersing medium. The liquid phase of the substance to be packaged is instantly frozen and now behaves as a solid that may be manipulated, including any particle size reduction operation, the same as any solid fine powder.

The present invention provides for packaging the medicament under a relatively high pressure of the packaging gas. Depending on the packaging gas used, a portion of the gas may remain in liquid form inside the package. Upon rupture in an appropriate inhaler, the powder material is aerosolized thus providing the fine distribution of powder required of inhalation therapy. Methods of sealing under pressure are well known to those skilled in the packaging of pharmaceuticals and foods.

FIG. 2 is a schematic illustration of a liquefied gas packaging station. Cryostat 15 is filled with liquefied gas 17, typically liquid nitrogen, from fill tube 23. A metered quantity of powdered medicament is introduced at powder loading port 19 and metered by metering valve or screw 21. The dispersion is well mixed using mixer 25, shown here as a motor driving an impeller blade. Evaporating nitrogen creates a positive pressure in the cryostat to drive the mixture through fill tube 27 to fill valve 29. As packaging blisters 31 are carried through the fill station by package transport 33, they are filled with a predetermined volume of liquid using metering valve 29. After filling, the liquid gas is allowed to partially evaporate and the blister sealed using thermocompression bonding or an adhesive.

Although blisters are shown in FIG. 2, capsules, thin plastic films, preformed pellets, linear and circular arrays of blister packs, semiconductor chips, etc. may also be filled or coated using the method of this invention.

If the package is sealed prior to the complete evaporation of liquid nitrogen, the gas pressure will build up in the sealed vessel. Since the density of gaseous nitrogen is less than 1/1,000 that of liquid nitrogen, most of the liquid nitrogen must be vaporized prior to sealing. Control of the package pressure may be carried out by using a closure lid that is held in place with a controlled pressure and when the internal package pressure becomes equal to the applied pressure, the closure lid may be thermocompression bonded, heat sealed, or crimped to form the package seal.

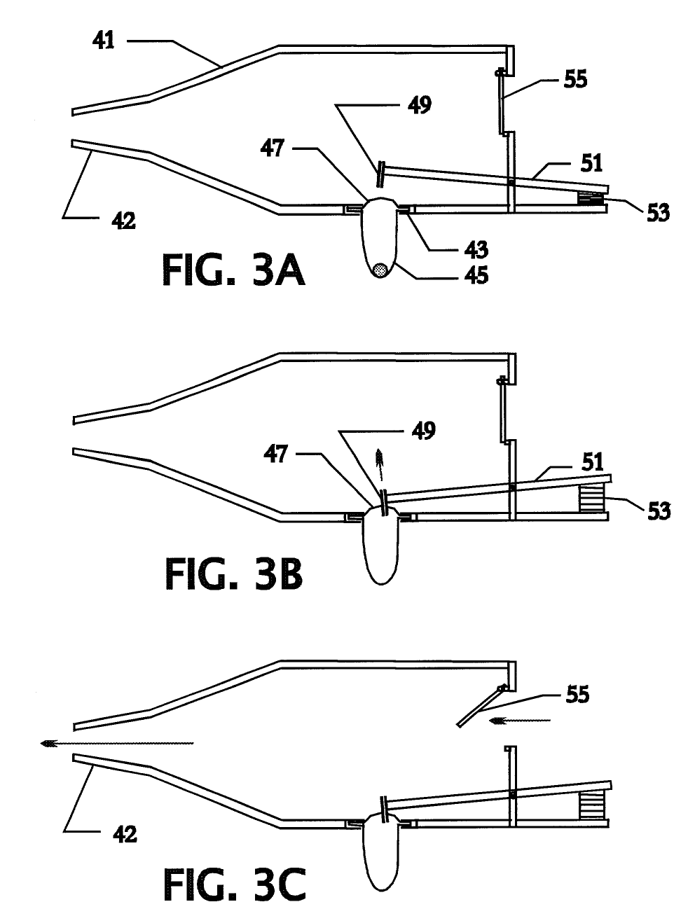

FIG. 3 schematically illustrates the use of a pressurized capsule in an inhaler. FIG. 3A shows the capsule loaded in the inhaler, FIG. 3B shows the capsule just ruptured with fine powder medicament escaping into the inhaler enclosure, and FIG. 3C depicts the inhaler operation during inhalation by the user. Inhaler housing 41 has three apertures; mouthpiece 42, capsule holder 43, and intake air valve 55. Capsule 45, including rupturable lid 47, is temporarily attached to the inhaler using attachment recess 43. The capsule is ruptured using piercing needle 49 that is held by arm 51. Arm 51 is compressed by spring 53 and held in place by a latching mechanism not shown.

In operation, the user compresses spring 53 by applying pressure to arm 51. A latch holds the spring in the compressed position. The user then loads a fresh capsule in the inhaler receptacle. Next, the user places his mouth over the mouthpiece and presses arm 51 latch release (not shown). This action causes the piercing needle to penetrate the capsule lid, releasing the powder into the inhaler chamber as a well-dispersed aerosol. The user then takes a deep breath and inhales the powder together with makeup air introduced by the opening previously closed by air intake valve 55. This intake valve is arranged to open only when the inhaler internal pressure is reduced by inhalation.

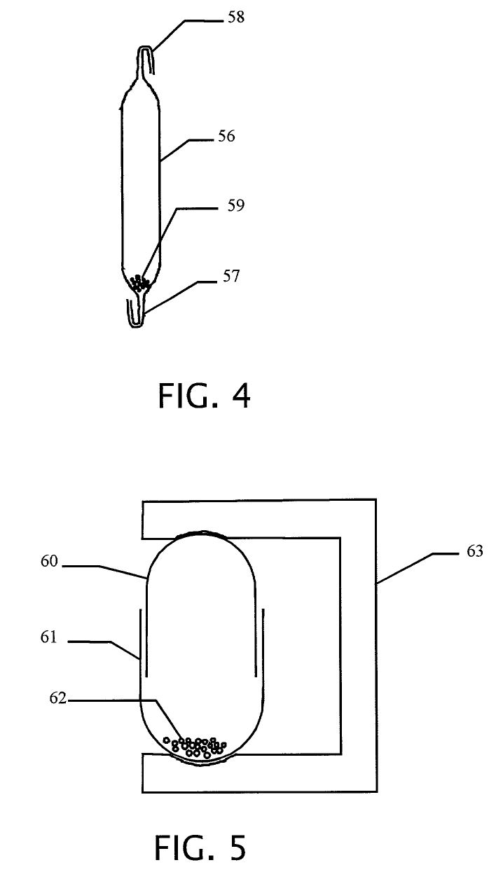

FIG. 4 and FIG. 5 are schematic sketches of two packages suitable for fine powder containment under high pressure.

FIG. 4 shows a sealed tube 56 containing powder 59. Each end of the tube is sealed by folding over the pinched ends and bonding the opposing surfaces as they are held in close contact. Seal 57 is formed prior to filling the tube with a measured quantity of liquid nitrogen containing the powder 59 to be packaged. Prior to evaporation of the entire load of liquid nitrogen, the upper seal 58 is formed.

One preferred construction employs an extruded aluminum tube having a finished wall thickness of 0.004 inches coated both internally and externally with a 0.004 inch thick thermoplastic material. The thermoplastic may be ethylene vinyl acetate copolymer, polyolefin, polyvinyl chloride polymer, polyethylene terephalate, or similar materials. The internal tube diameter depends upon the application and the dose load but may range from about two to about six millimeters. Tube length must be sufficient to provide a safe distance between the liquefied gas fill and the top seal 58 so that this seal may be thermally welded.

The package if formed by first pinching, folding, and thermally welding one tube end. A metered quantity of liquid nitrogen having a controlled concentration of the medicament powder is introduced into the tube through the open end. This step in the process may be carried out using a micropipette, syringe, or controlled flow apparatus. If required, a controlled portion of the liquid nitrogen may be allowed to evaporate. The top seal 58 is formed by pinching, folding, and welding the tube material.

Powder release in a suitable inhalation device may be carried out by puncture as shown in FIG. 3. Alternately, the tube may be bent or flexed to fracture the wall and release the powder, or the sealed end of the tube may be heated to soften the sealing compound, thus releasing the compressed contents.

Although plastic coated aluminum is one preferred material of construction, it should be obvious that many other high strength materials, including composites, may be employed in this packaging scheme.

FIG. 5 shows a packaging method employing a capsule. The larger diameter capsule component 61 is filled with a metered quantity of liquid nitrogen containing the desired dose of medicament powder 62. The smaller diameter capsule component 60 is then inserted into the lower component 61. Clip 63 is then inserted to hold the two capsule components in place. As the nitrogen evaporates, pressure is built up in the capsule. The clip prevents the capsule components from separating. A tight seal is formed as the pressure forces capsule component 60 tightly against the side of capsule component 61. The pressurized medicament is released when the clip is withdrawn from the capsule. Release of the clip may be made with the use of a wedge driven between the capsule and the clip upright.

The cost effectiveness of this filling process may be noted by considering the following example.

A 200-liter Dewar flask is filled with 200 liters of liquid nitrogen to which is added 5 grams of pharmaceutical powder to be ultimately employed in a dry powder inhaler. The mixture is thoroughly stirred to fully disperse the powder. A continuous line of blister packs is passed under a metering device that precisely transfers one milliliter of fluid into each pack. Each dose then contains 5/200,000 of a gram or 25 micrograms. The blister packs are filled in a moving line containing ten blister packs across the line width. Ten metering fill units are spaced across this line so that ten packs are filled simultaneously; each filled with a powder dry weight of 25 micrograms. Each fill unit is capable of filling 5 packs per second so that a total of 50 packs are filled each second or 180,000 packs are filled each hour. The packs may be sealed just as the last liquid nitrogen is vaporized so that the packs are then sealed in an inert nitrogen atmosphere under a slight positive pressure. This is a batch process and thus two complete fill systems must be employed to continuously operate a fill line. The cost of liquid nitrogen used in each unit dose preparation is about 4/100 of a cent.

While the invention has been described with reference to the structures disclosed herein, it is not confined to the details set forth and this application is intended to cover such modifications or changes as may come within the purposes of the improvements or the scope of the following claims.

What is claimed is:

1. A method of inhalation therapy comprising the steps of:
forming a unit dose of dry pharmaceutical powder in a pressurized airtight vessel,
attaching said airtight vessel to an inhaler,
rupturing said airtight vessel whereby an aerosol of said powder is released into said inhaler, and
inhaling said aerosol.

2. The method of claim 1 where said pressurized airtight vessel is pressurized by sealing said vessel while said vessel contains a small quantity of liquefied gas.

3. The method of claim 1 where said pressurized airtight vessel is pressurized by sealing said vessel while said vessel contains a small quantity of liquefied nitrogen.

4. The method of claim 1 where said pressurized airtight vessel is pressurized by sealing said vessel while said vessel contains a small quantity of liquefied fluorohydrocarbon.

5. The method of claim 1 where said pharmaceutical powder has a mean particle diameter between about 0.5 micrometers and about 5 micrometers.

6. The method of claim 1 where said aerosol is combined with inhaled air during said inhaling.

7. The method of claim 1 where said unit dose is between about 10 micrograms and 10 milligrams.

8. The method of claim 1 where said unit dose is comprised of pharmaceutical powders having a mean aerodynamic particle diameter of between about 0.5 microns and 5 microns.

9. The method of claim 1 where said liquefied gas is selected from the group of liquefied noble gases.

10. A method for pressurized packaging uniform small measures of a finely divided substance that comprises the steps of:
providing a unit dose package,
forming a mixture of said finely divided substance with a liquefied gas,
agitating said mixture to form a uniform dispersion,
dispensing a metered quantity of said uniform dispersion into said unit dose package, and
sealing said unit dose package to form an airtight chamber. whereby said sealed substrate is maintained under high pressure.

11. The method of claim 10 where said liquefied gas is liquid nitrogen.

12. The method of claim 10 where said liquefied gas is selected from the group of liquefied noble gases.

13. The method of claim 10 where said finely divided substance is selected from the group of pharmaceutical powders employed in inhalation therapy.

14. The method of claim 10 where said finely divided substance has a mean particle diameter between about 0.5 micrometers and about 5 micrometers.

15. The method of claim 10 where said unit dose package is in the form of a cylindrical tube sealed at one end.

16. The method of claim 10 where said unit dose package is in the form of a blister pack.

17. The method of claim 10 where said high pressure is between about 20 pounds per square inch and about 300 pounds per square inch.

18. A unit dose package for use in inhalation therapy consisting of a pressurized container containing one or more fine inhalable powders and a pressurized gas.

19. The unit dose package of claim 18 where said pressurized gas is nitrogen.

20. The unit dose package of claim 18 where said pressurized gas is at a pressure in excess of 20 pounds per square inch.
